(19) United States
(12) Patent Application Publication
(10) Pub. No.: US 2008/0228116 A1
(43) Pub. Date: Sep. 18, 2008

(54) Ergonomic universal sling

(76) Inventor: Michael K. Walker, Michigan City, IN (US)

Correspondence Address: Paul E Schaafsma;NovuslP, LLC, Suite 221, 521 West Superior Street, Chicago, IL, 60610-3135, US

(21) Appl. No.: 11/888,145

(22) Filed: Jul. 31, 2007

Related U.S. Application Data
(60) Provisional application No. 60/906,750, filed on Mar. 13, 2007.

(51) Int. Cl. A61F 5/00; A61F 13/00

(52) U.S. Cl. 602/4; 602/78

(57) ABSTRACT

A support sling is provided. A partial sleeve component is provided and adapted to be secured to the upper/opposing upper arm/shoulder area of a user, the partial sleeve component including hook or a loop adhesive fabric. A shoulder strap is provided, the shoulder strap including a hook or a loop adhesive fabric. The shoulder strap is adapted to connect to a load. In one embodiment, the shoulder strap can be secured to an arm sling adapted to cradle an arm of a user. The combined arm sling and the shoulder strap is placed over the head of the user, with the user's arm being placed in the arm sling, with the hook or loop adhesive fabric of the shoulder strap being strategically adhered to the hook or loop adhesive fabric of the partial sleeve component in a position designed to place and hold the injured arm and/or shoulder in the appropriate position for optimum healing advantages and enhanced pain management/comfort. In addition to bearing the weight of an arm, in further embodiments, the partial sleeve component and shoulder strap of the present invention can be utilized to bear other loads and transfer the load to a stronger, more capable part of the body such as, for example, carryalls such as personal travel bags, equipment bags, firearms, and other, as of yet unseen uses/advantages of secure sling technology.

### RELATED APPLICATIONS

This application is based upon U.S. Provisional Patent Application No. 60/906,750 titled “Super Sling” filed 13 Mar. 2007.

### FIELD OF THE INVENTION

The present invention relates to a device for supporting a load on a shoulder.

### BACKGROUND OF THE INVENTION

In the ordinary course of events, the shoulder bears substantially the full weight of the arm. The joint of the human shoulder provides for free movement of the arm; however, because of its construction, the shoulder joint is susceptible to injury. Injuries are commonplace in activities that require constant motion of the shoulder joint or subject the shoulder to stress. Once an injury to the shoulder area has occurred, it is frequently necessary to support the joint area to facilitate the healing process, prevent further damage, and minimize discomfort due to the injury.

In addition, the healing of other injuries requires that movement of the arm be immobilized. For example, a sprained or broken arm requires support of the arm in a position which will prevent strain on the shoulder joint of the injured arm. Restricting movement of the arm promotes rest and prevents further damage to bones or body tissues in the injured area.

One device that may be used in such cases is a sling that goes under and around the arm and then extends up and around the neck. In addition, a typical sling consists of a pouch into which an injured arm is placed and a strap that is worn around the shoulder and neck. In cases requiring that the arm be immobilized in close proximity to the body, an additional strap or swath can be placed around the arm and around the body, thus, holding the arm against the body and limiting motion.

Still, there remains a need for a support sling capable of bearing a load such as the weight of an arm and transferring this load to a stronger, more capable part of the body. It would be desirable to provide a support sling that readily supports the shoulder's typical load comfortably without stressing, fatiguing or possibly bruising/injuring the neck area. It would be desirable to providing a support sling that can be easily adjusted for each specific user.

### SUMMARY OF THE INVENTION

A support sling in accordance with the principles of the present invention bears a load such as the weight of the upper arm and transfers this load to a stronger, more capable part of the body by shifting the center of gravity outwardly from the neck to just above the shoulder joint area of the opposing arm. A support strap in accordance with the principles of the present invention supports the shoulder's typical load comfortably without stressing or fatiguing the neck area. A support sling in accordance with the principles of the present invention can be easily adjusted for each individual user.

In accordance with the principles of the present invention, a support sling is provided. A partial sleeve component is provided and adapted to be secured over the opposing upper-outer arm and extending to the top of the shoulder area of the user, the partial sleeve component including hook or a loop adhesive fabric. A shoulder strap is provided, with the shoulder strap including a hook or a loop adhesive fabric which the strap is then sandwiched securely between. The shoulder strap is adapted to connect quickly/easily to a load. In one embodiment, the shoulder strap can be secured to an arm sling adapted to cradle an arm of a user. The combined arm sling and the shoulder strap is placed over the head of the user, with the user's arm being placed in the arm sling, with the hook or loop adhesive fabric of the shoulder strap being strategically adhered to the hook or loop adhesive fabric of the partial sleeve component in a position designed to place and hold the injured arm and/or shoulder in the appropriate healing position. In addition to bearing the weight of an arm, in further embodiments, the partial sleeve component and shoulder strap of the present invention can be utilized to bear other loads and transfer the load to a stronger, more capable part of the body such as, for example, carryalls such as personal travel bags, equipment bags, firearms, baby pouches, tackle boxes, tool bags, bookbags, and the like.

### BRIEF DESCRIPTION OF THE DRAWINGS

FIG. 1 shows a pair of blanks that together form a partial sleeve component in accordance with the principles of the present invention.

FIG. 2 shows a shoulder strap in accordance with the principles of the present invention.

FIG. 3 shows one side of an adjustable release buckle.

FIG. 4 shows a pair of support straps in accordance with the principles of the present invention.

FIG. 5 shows blanks that can form an optional support patch in accordance with the principles of the present invention.

FIG. 6 shows a frontal view of the present invention on a user.

FIG. 7 shoes a side view of the present invention on a user.

FIG. 8 shows a view of an alternative embodiment of the present invention on a user.

FIG. 9 shoes a view of an alternative embodiment of the present invention on a user.

FIG. 10 shows another view of the alternative embodiment of FIG. 9.

FIG. 11 shoes another view of the alternative embodiment of FIG. 9.

### DETAILED DESCRIPTION OF THE INVENTION

The invention itself, together with further objects and attendant advantages, will be understood by reference to the following description, taken in conjunction with the accompanying drawings.

The present invention relates generally to a support sling for bearing a load such as the weight of an arm and transferring this load to a stronger, more capable part of the body. In one embodiment, the present invention relates to an arm sling having a partial sleeve component into which is secured a person's upper arm area and is placed so that the weight of the injured arm is supported directly on or just above the top/outward center of the opposing shoulder of the patient. In this embodiment, the present invention enhances stability by creating immobility for the injured area by completely restricting unwanted movements/motion. This decreases pain and discomfort and helps to better enable the healing process by helping prevent unwanted re-injuries due to unnecessary movements, through enhanced pain management.

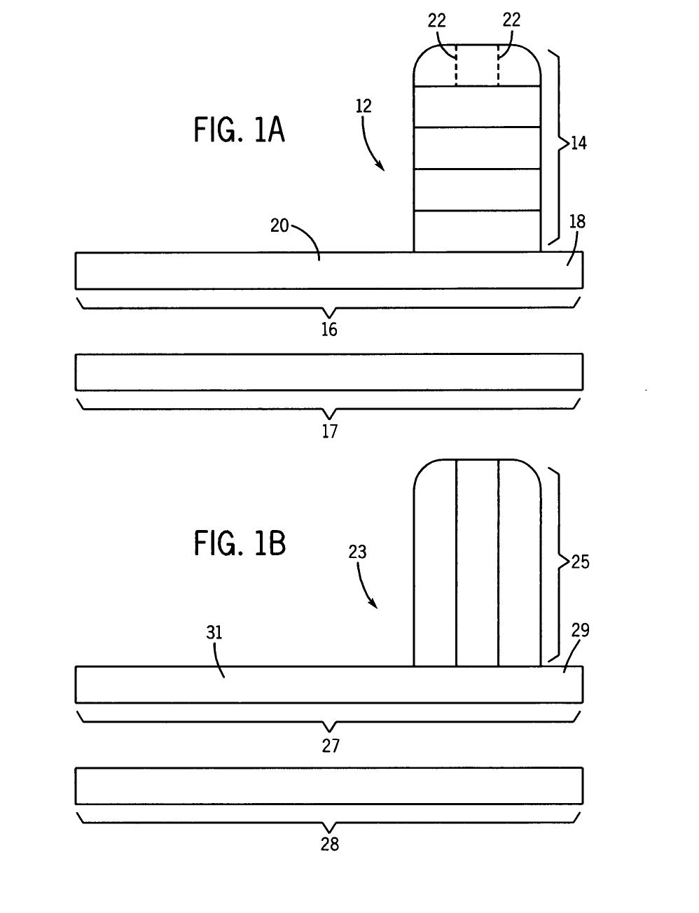

Initially, the various pieces of the support sling of the present invention are described. Referring to FIG. 1a, a first blank 12 can be formed from one of a hook or a loop adhesive fabric such as for example Velcro brand hook and loop fasteners available from Velcro USA Inc., 406 Brown Avenue, Manchester, N.H. 03103. The first blank 12 includes a main body portion 14 and an extended support portion 16. The extended support portion 16 can include a first projecting portion 18 that extends in one direction a relatively small extent beyond the outer periphery of the main body portion and, at an opposite side thereof, a second projecting portion 20 that extends in an opposite direction a relatively large extent. As described in detail below, the extended support 16 extends sufficiently such that it can be partially wrapped around the opposing upper arm/shoulder of a designated user.

In one embodiment, the extended support portion 16 can be formed integrally with the main body portion 14. In another embodiment, the extended support portion 16 can be formed separate from the main body portion 14. In yet another embodiment, a second, separate extended support portion 17 can be formed separate from the main body portion 14.

In one embodiment hereof, the main body portion 14 can be approximately 10 to 12 inches in length for a “large” aspect and approximately six inches in width; the extended support portion 16 can be approximately two inches in width; the first projecting portion 18 of the extended support portion 16 can extend beyond the outer periphery of the main body portion 14 approximately two inches; and the second projecting portion 20 of the extended support portion 16 can extend beyond the outer periphery of the main body portion 14 approximately 18 inches such that the total length of the extended support portion 16 is approximately 18 inches. Of course, from another perspective the main body portion 14 can be approximately 12 inches in length and approximately six inches in width, with the first projecting portion 18 extending beyond the outer periphery of the main body portion 14 approximately two inches and the second projecting portion 20 extending beyond the outer periphery of the main body portion 14 approximately 18 inches.

The upper periphery of the main body portion 14 can be rounded and can further be contoured to the shape of a shoulder. In one embodiment, the contour can be effectuated by cutting a plurality of slits 22 extending downwardly from the upper periphery of the main body portion 14 and overlapping the sides of these slits 22 together.

Referring to FIG. 1b, a second blank 23 can be formed from one of a cooperating hook or a loop adhesive fabric to act as an underlie to the first blank 12 depicted in FIG. 1 to thus form a partial sleeve component. Thus, the second blank 23 likewise includes a main body portion 25 and an extended support portion 27. The extended support portion 27 can include a first projecting portion 29 that extends in one direction a relatively small extent beyond the outer periphery of the main body portion and, at an opposite side thereof, a second projecting portion 31 that extends in an opposite direction a relatively large extent.

In one embodiment, the extended support portion 27 can be formed integrally with the main body portion 25. In another embodiment, the extended support portion 27 can be formed separate from the main body portion 25. In yet another embodiment, a second, separate extended support portion 28 can be formed separate from the main body portion 25.

In one embodiment hereof corresponding to the described embodiment of the first blank 12; the main body portion 25 can be approximately 10 to 12 inches in length for a “large” aspect and approximately six inches in width; the extended support portion 27 can be approximately two inches in width, the first projecting portion 29 of the extended support portion 27 can extend beyond the outer periphery of the main body portion 25 approximately two inches; and the second projecting portion 31 of the extended support portion 27 can extend beyond the outer periphery of the main body portion 25 approximately 18 inches such that the total length of the extended support portion 27 is approximately 18 inches. Again, from another perspective the main body portion 25 can be approximately 12 inches in length and approximately six inches in width, with first projecting portion 29 extending beyond the outer periphery of the main body portion 25 approximately two inches and the second projecting portion 31 extending beyond the outer periphery of the main body portion 25 approximately 18 inches.

Again, the upper periphery of the main body portion 25 can be rounded and can further be contoured to the shape of a shoulder. In one embodiment, the contour can be effectuated by cutting a plurality of slits extending downwardly from the upper periphery of the main body portion 25 and overlapping the sides of these slits together.

In one embodiment, the upper body portions 14, 25 can be comprises of a plurality of “horizontal” hook or a loop adhesive fabric bands secured together to form the upper body portion 14, 25 while the extended support portions 16, 27 can be an extended hook or a loop adhesive fabric band secured to the main body portion, as seen in FIG. 1a. In another embodiment, the upper body portions 14, 25 can be comprises of a plurality of “vertical” hook or a loop adhesive fabric bands secured together to form the upper body portion 14, 25 while the extended support portions 16, 27 can comprise a portion of the plurality of “vertical” hook or a loop adhesive fabric bands with the first projecting portions 18, 29 and the second projecting portions 20, 31 secured thereto, as seen in FIG. 1b. Of course, in additional embodiments each blank 12, 23 can be cut from a single sheet of hook or a loop adhesive fabric or various combinations thereof.

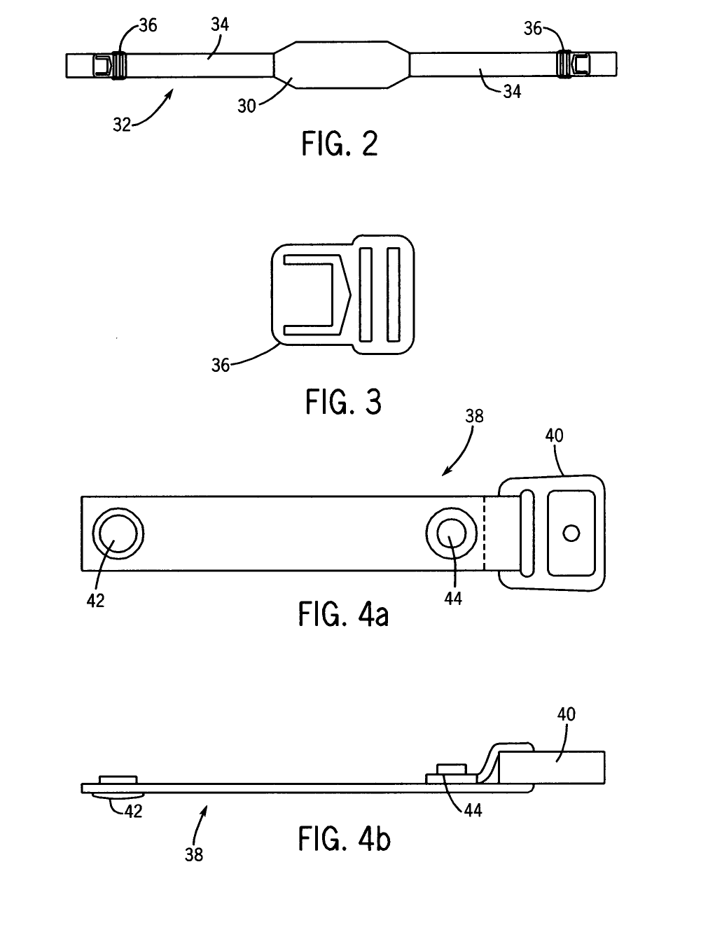

Referring to FIG. 2, a shoulder strap 32 is provided. The shoulder strap 32 includes a narrow band 34 made of a durable material such as for example nylon. In one embodiment, the narrow band 34 can be approximately one inch wide and approximately 46 inches in length. Sandwiched approximately in the center of the narrow band 34 is a pad 30 that comprises a hook or a loop adhesive fabric. In one embodiment, the pad 30 can be approximately 10 inches long and approximately two inches wide and can be tapered. The side of the pad 30 that faces downward during use is either a hook or loop adhesive fabric provided to adhesively cooperate with the a hook or loop adhesive fabric provided on the sleeve cover, as described in detail below. One side 36 of an adjustable release buckle as depicted in FIG. 3 is provided at each side of the pad 30 on the narrow band 34 for effective securing as described in detail below.

Referring to FIG. 4, two support straps 38 made of a durable material such as for example nylon are provided. FIG. 4a shows an overhead view of a first support strap 38 while FIG. 4b shows an elevated, side view of a second support strap 38. One end of each support strap 38 includes a cooperating side 40 of the adjustable release buckle seen in FIG. 3. Proximate the other end of each support strap 38, one side 42 of a heavy duty snap is provided, with a cooperating side 44 of the heavy duty snap provided proximate the cooperating side 40 of each adjustable release buckle.

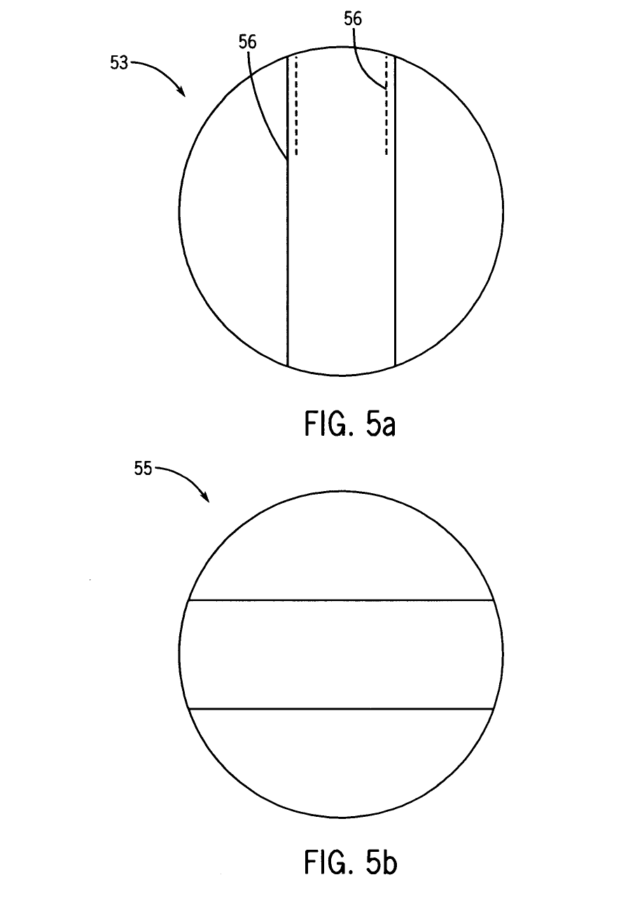

FIG. 5a shows a first support blank 53 that can be formed from one of a hook or a loop adhesive fabric and FIG. 5b shows a second support blank 55 that can be formed from the other of a hook or a loop adhesive fabric to act as an underlie to the first support blank 53 to form an optional support patch as described in detail below. In one embodiment, the first support blank 53 and the second support blank 55 can be round with an approximately a five inch diameter. The first support blank 53 and the second support blank 55 can be contoured to the shape of a shoulder. In one embodiment, the contour can be effectuated by cutting a plurality of slits 56 extending downwardly from the upper periphery of the first support blank 53 and the second support blank 55 and overlapping the sides of these slits together.

In one embodiment, the first support blank 53 can be comprised of a plurality of “vertical” hook or loop adhesive fabric bands secured together as seen in FIG. 5a and the second support blank 55 can be comprised of a plurality of “horizontal” hook or loop adhesive fabric bands secured together as seen in FIG. 5b. Of course, in an additional embodiment each blank 53, 55 can be cut from a single sheet of hook or loop adhesive fabric or any combinations thereof.

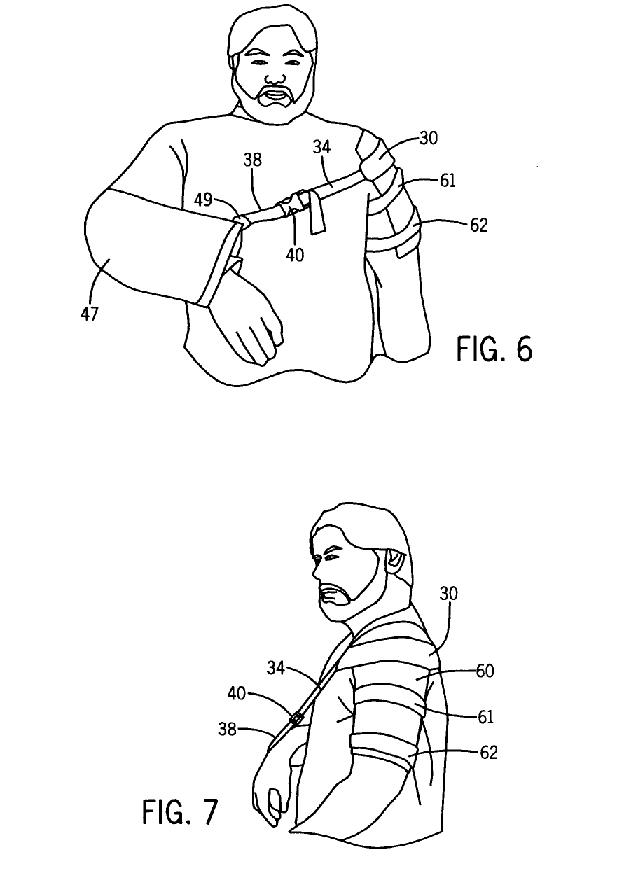

The present invention relates generally to a support sling for bearing a load. In one embodiment, the load can be the weight of an arm. The principles of the present invention can be applied to a plurality of designs of arm slings. Referring now to FIGS. 6 and 7, frontal and side views of one embodiment of the present invention on a user are seen. A standard envelope arm sling 47 is provided. The envelope arm sling 47 includes an open end through which a user's hand extends and a closed end wherein a user's elbow rests. A first support ring 49 is provided near the open end of the sling and a second support ring (not seen) is provided near the closed elbow side of the arm sling 47. As an alteration of a standard arm sling, in one embodiment a hook or a loop adhesive fabric area can be provided on the sides of the arm sling 47 to secure an optional support pillow as described in detail below.

To utilize the various pieces into the support sling of the present invention, the first blank 12 and the second blank 23 of FIG. 1 combine to form a partial sleeve component 60. The top of the partial sleeve component 60 is placed on the upper am/shoulder area of a users opposing arm, for example, the injured arm. By adjusting the overlap of the plurality of slits 22 extending downwardly from the upper periphery of the main body portion 14, 25, the partial sleeve component 60 can be adjusted to conform to different sized shoulders/persons. The extended support portions 16, 27 of the first blank 12 and the second blank 23 form a first arm support 62. The extended support portions 17, 28 of the first blank 12 and the second blank 23 form a second arm support 61. In the embodiments depicted in FIGS. 6-11, the first arm support 62 and the second arm support 61 are formed separate from the partial sleeve component 60. The first arm support 62 wraps partially around the arm of the user near the elbow and under the bicep area of the upper area of the un-injured arm and the second arm support 61 wraps partially around the arm of the user between the elbow and the shoulder while the top of the partial sleeve component 60 conforms to the top of opposing shoulder area.

One support strap 38 as seen in FIG. 4 is secured to the arm sling 47 for example by looping the strap around the support ring 49 provided near the hand opening of the sling and securing the two sides 42, 44 of the heavy duty snap together. The second support strap 38 as seen in FIG. 4 is likewise secured to the arm sling 47 for example by looping the strap around the support ring provided near the closed elbow side of the arm sling 47 and securing the two sides 42, 44 of the heavy duty snap together. Thus, the arm sling 47 is connected to the support straps 38 of FIG. 4, with the sides 40 of the adjustable release buckles ready to engage the cooperating sides 36 of the of the adjustable release buckles contained on the shoulder strap 32.

As previously described, the cooperating sides 36 of the adjustable release buckles are contained on each side of the shoulder strap 32 of FIG. 2. By adjusting the positions of the cooperating sides 36 of the adjustable release buckles on each side of the shoulder strap 32, the support sling can be adjusted to different sized users. The sides 36, 40 of the adjustable release buckles are connected, thus securing the arm sling 47 with the shoulder strap 32.

The combined arm sling 47 and the shoulder strap 32 which is then placed over the head of the user, with the user's arm being placed in the arm sling 47. To complete one embodiment of the present invention, the pad 34 of the shoulder strap 32 comprised of hook and loop adhesive fabric is strategically adhered to the partial sleeve component 60 is in a position designed to place and hold the injured arm and/or shoulder securely in the appropriate/most beneficial healing position.

As previously mentioned, in an additional embodiment support patch of FIG. 5 can be placed over the pad 34 of the shoulder strap 32 such that support patch is secured to both hook and loop adhesive fabrics of the shoulder strap 32 and the partial sleeve component 60 to provide additional, more effective securing. In addition, in an additional embodiment an optional, generally wedge-shaped, generally circular support pillow can be form-fitted to the underarm of the user on the injured side to help reduce unwanted movement and to help increase stability and comfort especially if recovering person has been fitted with wearer must support a cast. A hook or a loop adhesive fabric area can be provided slightly from center right on the support pillow can be used in cooperation with a hook or a loop adhesive fabric area provided on the arm sling to secure the support pillow firmly in place.

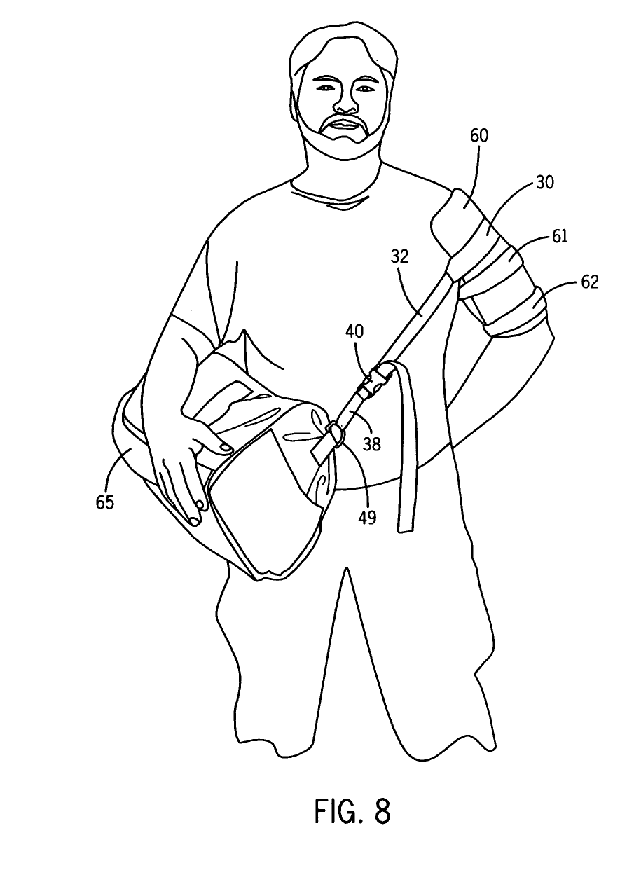

Thus, a support sling in accordance with the principles of the present invention bears a load such as the weight of an arm and transfers this load to a stronger, more capable part of the body to prevent further injury due to unwanted motions. In addition to bearing the weight of an arm, in further embodiments the partial sleeve component and shoulder strap of the present invention can be utilized to bear other loads and transfers the load to a stronger, more capable part of the body. Referring now to FIG. 8, in one such embodiment, the partial sleeve component and shoulder strap of the present invention can be used to support the weight of carryalls such as personal travel bags, or heavy equipment bag 65 on a user's shoulder.

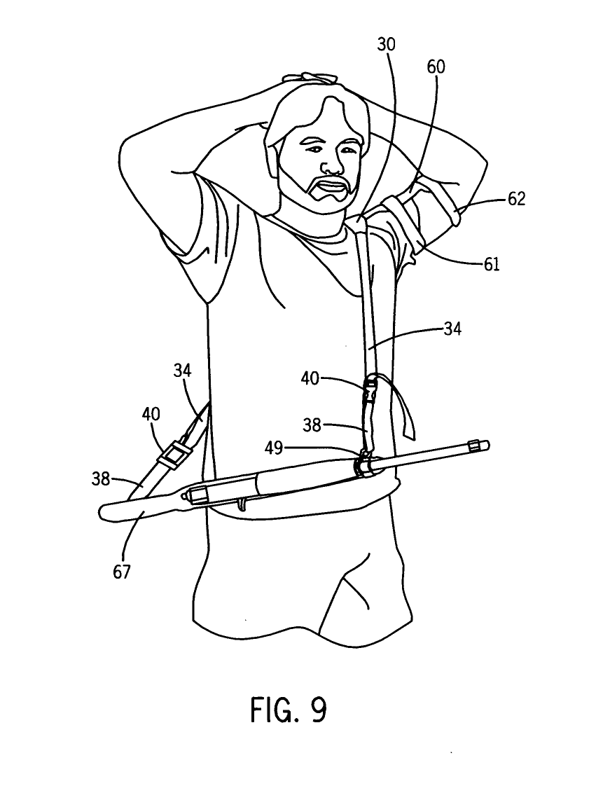

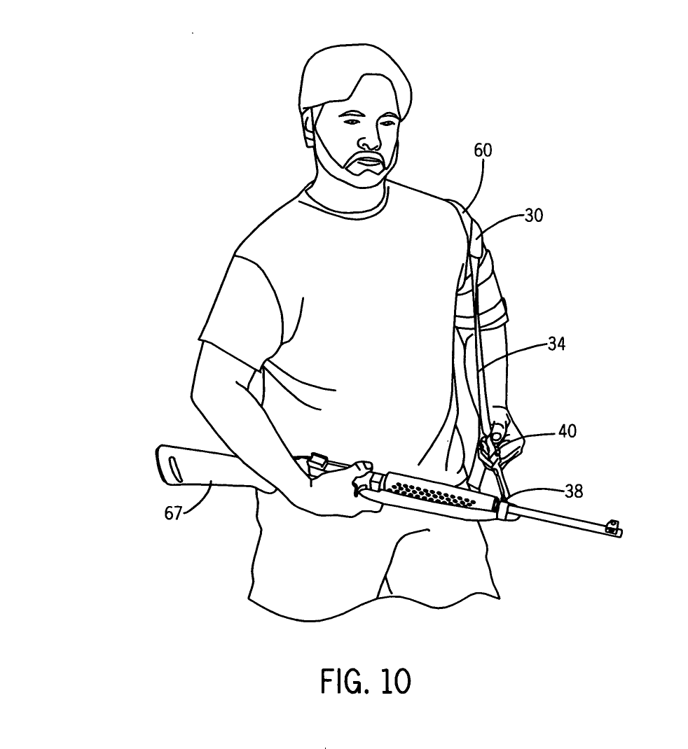

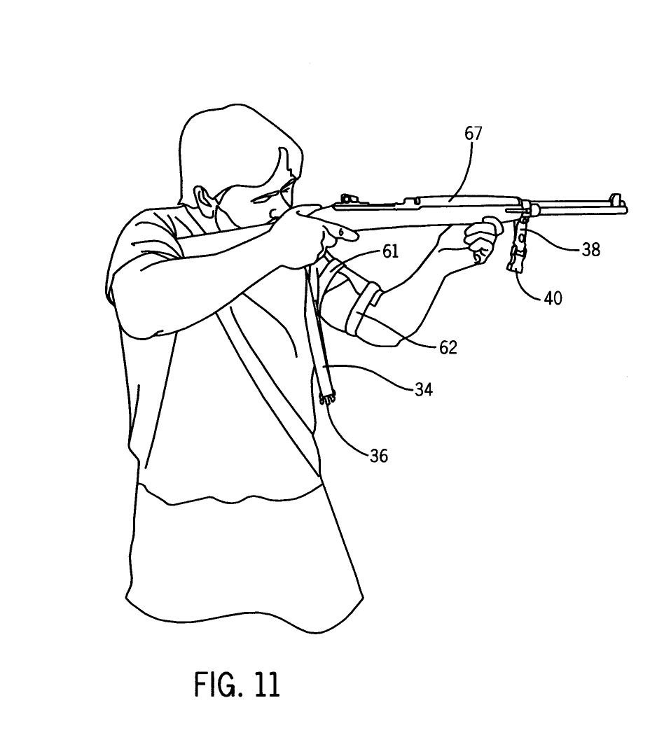

Referring now to FIGS. 9-11, in an embodiment representing a particular utility for the country's armed services, the partial sleeve component and shoulder strap of the present invention can be used to support the weight of a firearm 67 on a user's shoulder. When using the support sling of the present invention, the user can quickly release the sides 42, 44 of the adjustable release buckles as seen in FIG. 10, thus releasing the firearm 67 to enable the firearm 67 to be utilized, as seen in FIG. 11. This is particularly advantageous when the firearm is weighty such as for example a howitzer, a grenade launcher, or an M-60/M-50 machine gun, and 40 MM grenade launcher w/ammo, etc.

It should be understood that various changes and modifications preferred in to the embodiment described herein would be apparent to those skilled in the art. Such changes and modifications can be made without departing from the spirit and scope of the present invention and without diminishing its attendant advantages. It is therefore intended that such changes and modifications be covered by the appended claims as new uses become realized.

What is claimed is:

1. A support comprising:
a partial sleeve component adapted to be secured to the upper/opposing upper arm and shoulder of a user, the partial sleeve component providing a removable/securable component; and
a shoulder strap including a removable/securable fabric, the shoulder strap being adapted to connect to a load;
the shoulder strap and the partial sleeve component is removable and secured in such a way that a load connected to the shoulder strap is immovably secured and transferred to the opposing shoulder area of the user thereby altering center of gravity of the load to an area that can sustain it more comfortably than the neck area.

2. The support of claim 1 further wherein the removable securing process comprises hook or a loop adhesive fabric.

3. The support of claim 1 further wherein the partial sleeve component comprises a main body portion and an extended support portion, the extended support portion including a projecting portion that extends sufficiently to wrap partially around the opposing upper arm/shoulder of a user.

4. The support of claim 3 further wherein the main body portion is contoured to the shape of a shoulder.

5. The support of claim 1 further wherein the partial sleeve component comprises a first blank formed from one of a hook or a loop adhesive fabric and a second blank formed from one of a hook or a loop adhesive fabric that acts as an underlie to the first blank.

6. The support of claim 1 further wherein the shoulder strap comprises a narrow band and a pad comprised of a hook and a loop adhesive fabric provided to cooperate with a hook or loop adhesive fabric provided on the partial sleeve component.

7. The support of claim 6 further wherein the narrow band is comprised of nylon.

8. The support of claim 1 further wherein the shoulder strap is secured to the arm sling by a support strap secured by an adjustable release buckle.

9. The support of claim 1 further including a support patch adapted to be secured to the shoulder strap and the partial sleeve component.

10. The support of claim 1 further including an arm sling and further wherein the load is an arm.

11. The support of claim 1 further wherein the load is a bag.

12. The support of claim 1 further wherein the load is a firearm.

13. A support comprising:
a partial sleeve component adapted to be secured on the upper/opposing upper arm/shoulder of a user, the partial sleeve component including a main body portion and an extended support portion, the extended support portion including a projecting portion that extends sufficiently to partially wrap around the upper/opposing arm and conform to the top of the shoulder of a user; the partial sleeve component including a hook or a loop adhesive fabric; and
a shoulder strap including a hook or a loop adhesive fabric, the shoulder strap adapted to be removable and secured to the partial sleeve component.

14. The support of claim 13 further including an arm sling adapted to cradle the arm of a user, the shoulder strap secured to the arm sling wherein the combined arm sling and the removable/securable shoulder strap is placed over the head of a user, with a user's arm being placed in the arm sling, with the shoulder strap and the partial sleeve component removable but yet also securable.

15. The support of claim 2 further wherein the main body portion is contoured to the shape of a shoulder.

16. The support of claim 13 further wherein the partial sleeve component comprises a first blank formed from one of a hook or a loop adhesive fabric and a second blank formed from one of a hook or a loop adhesive fabric that acts as an underlie to the first blank.

17. The support of claim 13 further wherein the shoulder strap comprises a narrow band and a pad comprised of a hook and a loop adhesive fabric provided to cooperate with the hook or loop adhesive fabric provided on the partial sleeve component.

18. The support of claim 17 further wherein the narrow band is comprised of nylon.

19. The support of claim 13 further wherein the shoulder strap is secured to the arm sling by a support strap secured by an adjustable release buckle.

20. The support of claim 13 further including a support patch adapted to be secured to the shoulder strap and the partial sleeve component.

21. The support of claim 13 further including an arm sling and further wherein the load is an arm.

22. The support of claim 13 further wherein the shoulder strap is secured to a bag.

23. The support of claim 13 further wherein the shoulder strap is secured to a firearm.
